(19) United States
(12) Patent Application Publication
(10) Pub. No.: US 2008/0194466 A1
(43) Pub. Date: Aug. 14, 2008

(54) (5Z)-5-(6-Quinoxalinylmethylidene)-2-[(2,4,6-Trichlorophenyl)Amino]-1,3-Thiazol-4(5H)-One

(76) Inventor: Kevin J. Duffy, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/913,121

(22) PCT Filed: Jun. 8, 2006
(86) PCT No.: PCT/US06/22383

§ 371 Date: Oct. 30, 2007

Related U.S. Application Data
(60) Provisional application No. 60/688,676, filed on Jun. 8, 2005.

(51) Int. Cl. A61K 38/22; C07D 417/06; A61P 9/00; A61P 7/06; A61K 31/498

(52) U.S. Cl. 514/8; 544/353; 514/249

(57) ABSTRACT

Invented is the compound (5Z)-5-(6-quinoxalinylmethylidene)-2-[(2,4,6-trichlorophenyl)amino]-1,3-thiazol-4(5H)-one, and pharmaceutically acceptable salts, hydrates, solvates and pro-drugs thereof. Also invented are pharmaceutical compositions containing this compound, methods of preparing this compound and pharmaceutically acceptable salts, hydrates, solvates and pro-drugs thereof. Also invented are methods of using this compound as an inhibitor of hYAK3 proteins.

This invention relates to a novel compound useful for inhibiting hYAK3 proteins, specifically (5Z)-5-(6-quinoxalinylmethylidene)-2-[(2,4,6-trichlorophenyl)amino]-1,3-thiazol-4(5H)-one. This compound is represented by Structure I:

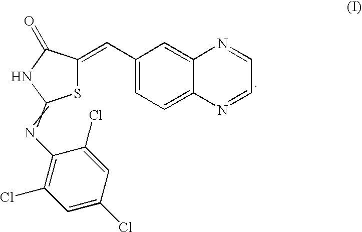

The compound of this invention is useful as an inhibitor of hYAK3 proteins and for treating or preventing diseases of the erythroid and hematopoietic systems, particularly anemias.

### DESCRIPTION OF THE RELATED ART

International Application No. PCT/US2003/037658, having an International filing date of Nov. 18, 2003; which also has International Publication Number WO 2004/047760 and an International Publication date of Jun. 10, 2004, describes a group of thiazolidinone compounds which are indicated as having hYAK3 inhibitory activity and which are indicated as being useful in the treatment of deficiencies in hematopoietic cells, in particular in the treatment of deficiencies in erythroid cells.

International Application No. PCT/US2003/037658 does not specifically disclose (5Z)-5-(6-quinoxalinylmethylidene)-2-[(2,4,6-trichlorophenyl)amino]-1,3-thiazol-4(5H)-one.

### DETAILED DESCRIPTION OF THE INVENTION

The present invention is concerned with the novel compound (5Z)-5-(6-quinoxalinylmethylidene)-2-[(2,4,6-trichlorophenyl)amino]-1,3-thiazol-4(5H)-one (hereinafter—“Compound A”), pharmaceutically acceptable salts, hydrates, solvates and pro-drugs of this compound, processes for its preparation, pharmaceutical formulations comprising this compound as an active ingredient, and methods for treating or preventing diseases of the erythroid and hematopoietic systems with Compound A, and/or a pharmaceutically acceptable salt, hydrate, solvate or pro-drug thereof, or a pharmaceutical formulation thereof.

It has been found that Compound A is advantageous over closely related compounds in International Application No. PCT/US2003/037658. The presently invented Compound A has significantly greater bioavailability in vivo over the most closely related compounds in International Application No. PCT/US2003/037658.

While the thiazolidinone compounds disclosed in International Application No. PCT/US2003/037658 are useful as inhibitors of hYAK3 proteins, particularly in the treatment of deficiencies in hematopoietic cells in particular in the treatment of deficiencies in erythroid cells, Compound A has the added advantage of enhanced bioavailability.

The compound of this invention, Compound A, is useful as an inhibitor of the hYAK3 proteins, particularly for treating or preventing diseases of the erythroid and hematopoietic systems. Compound A can be administered in a conventional dosage form prepared by combining Compound A, and/or a pharmaceutically acceptable salt, hydrate, solvate or pro-drug thereof, with a conventional pharmaceutically acceptable carrier or diluent according to techniques readily known to those of skill in the art, such as those described in International Application No. PCT/US2003/037658. The route of administration may be oral, parenteral or topical. The term parenteral as used herein includes intravenous, intramuscular, subcutaneous, intranasal, intrarectal, intravaginal or intraperitoneal administration. Oral administration is generally preferred.

As used herein, the term “effective amount” means that amount of Compound A that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “treating” and derivatives thereof as used herein, is meant prophylactic and therapeutic therapy.

As used herein, the crisscrossed double bond indicated by the symbol [symbol] denotes Z and/or E stereochemistry around the double bond. In other words Compound A can be either in the Z or E stereochemistry around this double bond, or Compound A can also be in a mixture of Z and E stereochemistry around the double bond. Further, Compound A may exist in one tautomeric form or in a mixture of tautomeric forms. An example of one alternative tautomeric form is shown below.

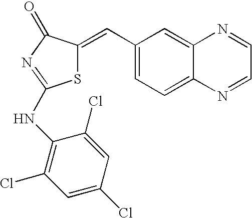

The present invention contemplates all possible tautomeric forms.

By the term “co-administering” and derivatives thereof as used herein is meant either simultaneous administration or any manner of separate sequential administration of Compound A, and/or a pharmaceutically acceptable salt, hydrate, solvate or pro-drug thereof, and a further active ingredient or ingredients, known to be useful in treating diseases of the erythroid and hematopoietic systems, particularly anemias, including EPO or a derivative thereof. The term further active ingredient or ingredients, as used herein, includes any compound or therapeutic agent known to or that demonstrates advantageous properties when administered to a patient in need of treatment for diseases of the erythroid and hematopoietic systems, particularly anemias. Preferably, if the administration is not simultaneous, the compounds are administered in a close time proximity to each other. Furthermore, it does not matter if the compounds are administered in the same dosage form, e.g. one compound may be administered topically and another compound may be administered orally.

Because the novel compound of the present invention is active as a hYAK3 inhibitor it exhibits therapeutic utility in treating diseases of the erythroid and hematopoietic systems, including but not limited to, anemias due to renal insufficiency or to chronic disease, such as autoimmunity, HIV, or cancer, and drug-induced anemias, myelodysplastic syndrome, aplastic anemia, myelosuppression, and cytopenia.

Compound A is useful in treating diseases of the erythroid and hematopoietic systems, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection.

Compound A is also useful for enhancing normal red blood cell numbers. Such enhancement is desirable for a variety of purposes, especially medical purposes such as preparation of a patient for transfusion and preparation of a patient for surgery.

Compound A is tested for its ability to inhibit the hYAK3 kinase enzyme by known methods such as those described in International Application No. PCT/US2003/037658.

When tested in in vitro assays for hYAK3 kinase enzyme inhibition, Compound A exhibited an activity similar to Compound B (described herein) and Compound C (described herein).

The pharmaceutically active compound of this invention is useful as a hYAK3 inhibitor in mammals, particularly humans, in need thereof.

The present invention therefore provides a method of treating diseases of the erythroid and hematopoietic systems, particularly anemias and other conditions requiring hYAK3 inhibition, which comprises administering an effective amount of Compound A, and/or a pharmaceutically acceptable salt, hydrate, solvate or pro-drug thereof. Compound A also provides for a method of treating the above indicated disease states because of its ability to act as a hYAK3 inhibitor. The drug may be administered to a patient in need thereof by any conventional route of administration, including, but not limited to, intravenous, intramuscular, oral, subcutaneous, intradermal, and parenteral.

The pharmaceutically active compound of the present invention is incorporated into a convenient dosage form such as a capsule, tablet, or injectable preparation. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented Compound A in a pharmaceutical dosage unit as described above will be an efficacious, nontoxic quantity preferably selected from the range of 0.001-100 mg/kg of total body weight, preferably 0.001-50 mg/kg. When treating a human patient in need of hYAK3 inhibition, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of Compound A, most preferably from 0.5 to 1,000 mg of Compound A. Oral administration, which uses lower dosages is preferred. Parenteral administration, at high dosages, however, also can be used when safe and convenient for the patient. The above dosages relate to the preferred amount of Compound A expressed as the free acid.

It will be recognized by one of skill in the art that the optimal quantity and spacing of individual dosages of Compound A will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of Compound A given per day for a defined number of days, can be ascertained by those skilled in the art using conventional course of treatment determination tests.

The method of this invention of inducing hYAK3 inhibitory activity in mammals, including humans, comprises administering to a subject in need of such activity an effective hYAK3 inhibiting amount of Compound A, and/or a pharmaceutically acceptable salt, hydrate, solvate or pro-drug thereof.

The invention also provides for the use of Compound A, and/or a pharmaceutically acceptable salt, hydrate, solvate or pro-drug thereof, in the manufacture of a medicament for use as a hYAK3 inhibitor.

The invention also provides for the use of Compound A, and/or a pharmaceutically acceptable salt, hydrate, solvate or pro-drug thereof, in the manufacture of a medicament for use in therapy.

The invention also provides for the use of Compound A, and/or a pharmaceutically acceptable salt, hydrate, solvate or pro-drug thereof, in the manufacture of a medicament for use in treating diseases of the erythroid and hematopoietic systems, particularly anemias.

The invention also provides for a pharmaceutical composition for use as a hYAK3 inhibitor which comprises Compound A, and/or a pharmaceutically acceptable salt, hydrate, solvate or pro-drug thereof, and a pharmaceutically acceptable carrier.

The invention also provides for a pharmaceutical composition for use in the treatment of diseases of the erythroid and hematopoietic systems, particularly anemias, which comprises Compound A, and/or a pharmaceutically acceptable salt, hydrate, solvate or pro-drug thereof, and a pharmaceutically acceptable carrier.

No unacceptable toxicological effects are expected when the compound of the invention is administered in accordance with the present invention.

In addition, the pharmaceutically active compound of the present invention can be co-administered with further active ingredients, such as other compounds known to treat diseases of the erythroid and hematopoietic systems, particularly anemias, or compounds known to have utility when used in combination with a hYAK3 inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXAMPLE 1

### Preparation of

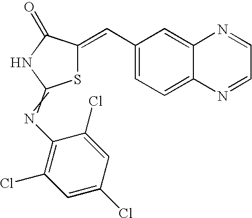

### (5Z)-5-(6-quinoxalinylmethylidene)-2-[(2,4,6-trichlorophenyl)amino]-1,3-thiazol-4(5H)-one

a) 6-Methylquinoxaline. A suspension of 3,4-diaminotoluene (50.0 g; 0.409 mol.) and glyoxal (40% aq. soln.; 52.0 mL; 0.450 mol.) in water (150 mL) and CH₃CN (20.0 mL) was heated to 60° C. for 1 h. Heating was then discontinued and brine (100 mL) was added. The solution was extracted with EtOAc (3×150 mL) and the combined organic layers were dried over MgSO₄, filtered, and concentrated in vacuo. Purification via distillation under reduced pressure (120° C., 10 torr) provided 6-methylquinoxaline (48.0 g, 81%) as a clear, colorless oil. ¹H NMR (400 MHz, CDCl₃) δ ppm 2.61 (s, 3H) 7.61 (dd, J=8.59, 1.77 Hz, 1H) 7.88 (s, 1H) 8.00 (d, J=8.59 Hz, 1H) 8.79 (dd, J=9.85, 1.77 Hz, 2H) MS (ES+) m/e 145 [M+H]⁺.

b) Quinoxaline-6-carbaldehyde. A suspension of 6-methylquinoxaline (8.0 g; 0.055 mol.) and selenium dioxide (6.77 g; 0.061 mol.) in 1,4-dioxane (5.0 mL) was irradiated at 200° C. for 30 min. in a Biotage Initiator microwave synthesizer. The above procedure was repeated five further times and the combined, cooled reaction mixtures were dissolved in CH₂Cl₂, filtered through a plug of celite, and concentrated in vacuo. Purification via flash column chromatography (silica gel, 20-50% ethyl acetate in hexanes) followed by crystallization from CH₂Cl₂ provided quinoxaline-6-carbaldehyde (40.0 g, 91%) as a white solid. ¹H NMR (400 MHz, CDCl₃) [symbol] ppm 10.25 (s, 1H) 8.95 (s, 2H) 8.57 (d, J=1.3 Hz, 1H) 8.24 (dd, J=8.6, 1.5 Hz, 1H) 8.20 (d, J=8.6 Hz, 1H). MS (ES+) m/e 159 [M+H]⁺.

c) 2-[(2,4,6-Trichlorophenyl)amino]-1,3-thiazol-4(5H)-one. A solution of N-(2,4,6-trichlorophenyl)thiourea (Aldrich Chemical Company, Milwaukee) (5.2 g; 0.02 mol.) and chloroacetic acid (2.6 g; 0.022 mol.) in glacial acetic acid (95.0 mL) was stirred and heated under reflux for 4 h. The heating source was then removed and, while cooling, water (200 mL) was slowly added dropwise. The mixture was filtered when the temperature reached 40° C. to give the title compound (4.0 g; 68%) as a colorless solid. ¹H NMR (400 MHz, DMSO-d₆) δ ppm 4.12 (s, 2H) 7.71 (s, 2H) 12.29 (s, 1H).

d) (5Z)-5-(6-Quinoxalinylmethylidene)-2-[(2,4,6-trichlorophenyl)amino]-1,3-thiazol-4(5″-one, sodium salt, 0.5 hydrate. A solution of the compound from Example 1c) (295 mg; 1.0 mmol.), quinoxaline-6-carbaldehyde from 1b) (158 mg; 1.0 mmol.) and piperidine (0.11 mL; 1.1 mmol.) in ethanol (2.5 mL) was stirred and heated at 150° C. for 30 min. in a Biotage Initiator microwave synthesizer. The reaction mixture was then cooled, poured into 1M aqueous hydrochloric acid (50.0 mL) and filtered to give the title compound (286 mg; 66%) as a yellow powder. ¹H NMR (400 MHz, DMSO-d₆) δ ppm 7.81 (s, 2H) 7.98 (dd, J=8.59, 1.52 Hz, 1H) 8.01 (s, 1H) 8.18 (d, J=8.59 Hz, 1H) 8.24 (d, J=1.26 Hz, 1H) 8.99 (s, 2H) 13.17 (s, 1H). This material was further purified by crystallization from water (5.0 mL)/1M aqueous sodium hydroxide (1.0 mL)/ethanol (1.0 mL) to afford the monosodium salt, hemihydrate of the title compound (201 mg; 46%) as bright yellow needles. C₁₈H₈N₄OSCl₃Na.0.5H₂O requires: % C, 46.3; % H, 1.9; % N, 12.0; found: % C, 46.3; % H, 2.1; % N, 11.8.

### EXAMPLE 2

### Bioavailability

The bioavailability of the sodium salt of Compound A: (5Z)-5-(6-quinoxalinylmethylidene)-2-[(2,4,6-trichlorophenyl)amino]-1,3-thiazol-4(5H)-one sodium salt, was compared to what is considered to be the two most closely related compounds prepared in International Application No. PCT/US2003/037658. The first is the sodium salt of the compound of example 23 in International Application No. PCT/US2003/037658: 2-(2,6-dichloro-phenylimino)-5-(quinolin-6-ylmethylene)-thiazolidin-4-one sodium salt,

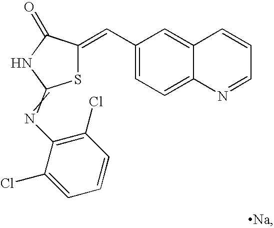

hereinafter (Compound B). The second is the sodium salt of the compound of example 26 in International Application No. PCT/US2003/037658: 2-(2-chloro-phenylimino)-5-(quinoxalin-6-ylmethylene)-thiazolidin-4-one sodium salt,

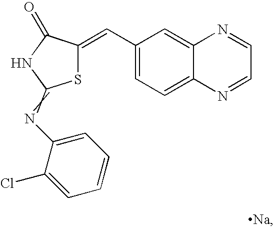

hereinafter (Compound C).

Compounds B and C can be prepared as described in International Application No. PCT/US2003/037658. The sodium salts of these compounds are prepared by methods well known in the art, such as described in Example 1d) above.

The sodium salt of Compound A was fed to male Sprague-Dawley rats by oral gavage in a formulation with 50% PEG-400, 10% ethanol, 40% of 40% (w/v) aqueous Encapsin in water at a dose of between 1 and 4 mg/kg (16 mL of dose solution per kg). Blood (120 microliters) was sampled at the following time intervals: 0, 20, 40, 60, 120, 180, 240, 360, 480, and 1440 min. The concentration of Compound A was quantified by LC/MS/Ms analysis of an aliquot (25 microliters blood+25 microliters water) of these samples and the overall blood exposure reported as the Dose-Normalised Area Under the Curve (DNAUC) from a concentration versus time plot and expressed in the units microgram hours per milliliter per minute per kilogram (ug.h/mL/mg/kg). The oral exposures of the sodium salt of Compound B and the sodium salt of Compound C were quantified by the same method.

The data are summarized in Table 1 below.

TABLE 1

| Dose (~1-4 mg/kg) | | | Compound |
|---|---|---|---|
| Oral DNAUC rats | Compound A•Na | Compound B•Na | C•Na |
| (Sprague-Dawley) | mg/ml | mg/ml | mg/ml |
| (ug · h/mL/min/kg) | 26.11 | 0.49 | 0.34 |

The present invention includes within its scope pharmaceutical compositions comprising (5Z)-5-(6-quinoxalinylmethylidene)-2-[(2,4,6-trichlorophenyl)amino]-1,3-thiazol-4(5H)-one, and/or pharmaceutically acceptable salts, hydrates, solvates or pro-drugs thereof, as the active ingredient, in association with a pharmaceutically acceptable carrier or diluent. The compound of this invention can be administered by oral or parenteral routes of administration and can be formulated in dosage forms appropriate for each route of administration including capsules, tablets, pills, powders and granules. In such solid dosage forms, the active ingredient is admixed with at least one inert diluent. The oral dosage forms can also comprise, as is normal practice, additional substances other than inert diluents, e.g., lubricating agents, glidants and antioxidants. In the case of capsules, tablets and pills, the dosage forms may also comprise buffering agents. Tablets and pills can additionally be prepared for a sustained release.

Preparations according to this invention for parenteral administration include sterile aqueous solutions although nonaqueous suspensions of emulsions can be employed. Such dosage forms may also contain adjuvants such as preserving, wetting, osmotic, buffering, emulsifying and dispersing agents. They may be sterilized by, for example, filtration through a bacteria retaining filter, by incorporating sterilizing agents into the compositions, irradiating the compositions or by heating the compositions.

The following examples further illustrate the pharmaceutical compositions which are a feature of this invention.

### EXAMPLE 4

### Tablet Composition

Lactose, microcrystalline cellulose, sodium starch glycolate, magnesium stearate and (5Z)-5-(6-quinoxalinylmethylidene)-2-[(2,4,6-trichlorophenyl)amino]-1,3-thiazol-4(5H)-one are blended in the proportions shown in Table 2 below. The blend is then compressed into tablets.

TABLE 2

| INGREDIENT | mg. |
|---|---|
| (5Z)-5-(6-quinoxalinylmethylidene)-2- | 8 |
| [(2,4,6-trichlorophenyl)amino]-1,3- | |
| thiazol-4(5H)-one | |
| microcrystalline cellulose | 112 |
| lactose | 70 |
| sodium starch glycolate | 8 |
| magnesium stearate | 2 |

### EXAMPLE 5

### Injectable Parenteral Composition

An injectable form for administering (5Z)-5-(6-quinoxalinylmethylidene)-2-[(2,4,6-trichlorophenyl)amino]-1,3-thiazol-4(5H)-one is produced by stirring 5.0 mg. of the compound in 1.0 ml. of normal saline.

### EXAMPLE 6

### Capsule Composition

An oral dosage form for administering the present invention is produced by filing a standard two piece hard gelatin capsule with the ingredients in the proportions shown in Table 3, below.

TABLE 4

| INGREDIENTS | AMOUNTS |
|---|---|
| (5Z)-5-(6-quinoxalinylmethylidene)-2-[(2,4,6- | 25 mg |
| trichlorophenyl)amino]-1,3-thiazol-4(5H)-one | |
| Lactose | 55 mg |
| Talc | 16 mg |
| Magnesium Stearate | 4 mg |

While the preferred embodiments of the invention are illustrated by the above, it is to be understood that the invention is not limited to the precise instructions herein disclosed and that the right to all modifications coming within the scope of the following claims is reserved.

What is claimed is:

1. The compound (5Z)-5-(6-quinoxalinylmethylidene)-2-[(2,4,6-trichlorophenyl)amino]-1,3-thiazol-4(5″-one.

2. A pharmaceutically acceptable salt, hydrate, solvate or pro-drug of the compound of claim 1.

3. The monosodium salt of the compound of claim 1.

4. The monosodium salt according to claim 3 as a hydrate.

5. The compound of claim 4 where the hydrate contains less than 5.25 waters of solvation.

6. A pharmaceutical composition comprising the compound of claim 1 and a pharmaceutically acceptable carrier.

7. A pharmaceutical composition comprising a pharmaceutically acceptable salt, hydrate, solvate or pro-drug of the compound of claim 1 and a pharmaceutically acceptable carrier.

8. A process for preparing a pharmaceutical composition containing a pharmaceutically acceptable carrier or diluent and an effective amount of the compound of claim 1, which process comprises bringing the compound of claim 1 into association with a pharmaceutically acceptable carrier or diluent.

9. A process for preparing a pharmaceutical composition containing a pharmaceutically acceptable carrier or diluent and an effective amount of a pharmaceutically acceptable salt, hydrate, solvate or pro-drug of the compound of claim 1, which process comprises bringing the pharmaceutically acceptable salt, hydrate, solvate or pro-drug of the compound of claim 1, into association with a pharmaceutically acceptable carrier or diluent.

10. A method of inhibiting hYAK3 in a mammal; comprising, administering to the mammal a therapeutically effective amount of the compound of claim 1, or a pharmaceutically acceptable salt, hydrate, solvate or pro-drug thereof.

11. The method of claim 10 wherein the mammal is a human.

12. A method of treating or preventing deficiencies in hematopoietic cells, in particular in the treatment of deficiencies in erythroid cells, by administering to a mammal a therapeutically effective amount of the compound of claim 1, or a pharmaceutically acceptable salt, hydrate, solvate or pro-drug thereof and one or more of pharmaceutically acceptable: carriers, diluents and excipients.

13. A method of claim 12 in which deficiencies of the erythroid and hematopoietic systems are selected from the group consisting of: anemia, aplastic anemia, myelodysplastic syndrome, myelosuppression, and cytopenia.

14. A method of treating or preventing diseases selected from the group consisting of: anemia, aplastic anemia, myelodysplastic syndrome, myelosuppression, and cytopenia; comprising, administering to a mammal a therapeutically effective amount of the compound of claim 1, or a pharmaceutically acceptable salt, hydrate, solvate or pro-drug thereof and one or more of pharmaceutically acceptable: carriers, diluents and excipients.

15. The method of claim 12 wherein the mammal is a human.

16. A method of treating deficiencies of the hematopoietic system, in a mammal in need thereof, which comprises: administering to such mammal a therapeutically effective amount of
a) the compound claim 1 and/or a pharmaceutically acceptable salt, hydrate, solvate or pro-drug thereof; and
b) EPO or a derivative thereof.
